SEQUENCE LISTING (2 sequences)

SEQ ID NO: 1 - protein, 12 aa, Artificial Sequence
  TfR/CD71 targeting peptide
THRPPMWSPVWP

SEQ ID NO: 2 - DNA, 36 bp, Artificial Sequence
  Capture Oligonucleotide Complement
ttctcgtgttccgtttgtactctaaggtggattttt

(19) United States
(12) Patent Application Publication
(10) Pub. No.: US 2017/0209599 A1
(43) Pub. Date: Jul. 27, 2017

(54) Functionalized DNA Dendrimers For Gene Delivery To Cells

(71) Applicants: Genisphere, LLC, Hatfield, PA (US); Lankenau Institute of Medical Research, Wynnewood, PA (US)

(72) Inventors: Robert C. Getts, Collegeville, PA (US); Janet Sawicki, Newtown Square, PA (US)

(21) Appl. No.: 15/454,612

(22) Filed: Mar. 9, 2017

Related U.S. Application Data
(63) Continuation of application No. 14/764,388, filed on Jul. 29, 2015, now abandoned.
(60) Provisional application No. 61/759,558, filed on Feb. 1, 2013.

(51) Int. Cl. A61K 48/00; A61K 47/10; A61K 47/36; C12N 9/10; A61K 38/45

(52) U.S. Cl. CPC A61K 48/0058; C12N 9/1077; C12Y 204/02036; A61K 38/45; A61K 48/0075; A61K 47/36; A61K 47/10

(57) ABSTRACT

Compositions are disclosed which comprise a DNA dendrimer having one or more DNA sequences linked thereto, the DNA sequences comprising DNA sequences encoding a polypeptide or regulatory RNA linked to DNA sequences that regulate expression of the DNA sequences encoding a polypeptide or regulatory RNA to produce an RNA coding for the polypeptide or to produce the regulatory RNA. Also disclosed are methods for treating diseases and conditions of cells by delivering the dendrimers to the cells, with subsequent expression of the encoded polypeptide or regulatory RNA.

### CROSS REFERENCE TO RELATED APPLICATIONS

This is a continuation of U.S. application Ser. No. 14/764,388, filed Jul. 29, 2015, which is a National Phase entry of International Application No. PCT/US2014/014104, filed Jan. 31, 2014, which claims priority to U.S. Provisional Application No. 61/759,558, filed Feb. 1, 2013, the disclosures of which are incorporated herein by reference in their entireties.

### REFERENCE TO SEQUENCE LISTING

The material contained in the text file identified as DSC0054-00WO_ST25.txt (created Apr. 15, 2014, 773 bytes) is hereby incorporated by reference.

### TECHNICAL FIELD

The present invention relates to the field of functionalized DNA dendrimers for delivery of genes to cells, and expression of the genes in the cells.

### BACKGROUND

Gene therapy is a promising treatment for a variety of cellular abnormalities, diseases and conditions because delivery of therapeutic DNA allows for direct treatment of the affected cell. In theory, the ability to target delivery of the DNA to the desired cell, and to control expression once the DNA is taken up by the cell, should provide an improved level of therapeutic efficiency and specificity that is difficult to achieve using conventional therapies. However, in practice, the potential of DNA therapies has not been fully realized. One of the challenges has been the choice of an appropriate vector for delivery of DNA to cells. Viral vectors have been associated with serious collateral effects. As a result, nonviral vectors have been sought. Cationic liposomes have been investigated for gene transfer into cells but have not provided satisfactory results.

More recently, nanoparticles formed by complexing the encoding DNA with a cationic polymer have shown promise for gene therapy, particularly when functionalities are added to improve delivery and therapeutic efficacy (e.g., targeting moieties to improve cell specificity and accumulation, and membrane-permeation moieties to improve cell uptake, and features to prevent degradation). Many of these functionalities are considered necessary for delivery of therapeutic biologics to cells, including genes. Unfortunately, they also increase the complexity of the gene therapeutic and the cost of producing it. In addition, molecular weight, size, charge after DNA condensation and choice of end group termination can have large and unpredictable effects on the transfection potential of the nanoparticle. Further, cationic polymer nanoparticles often lose their DNA delivery capabilities upon conjugation to targeting moieties that would otherwise improve their specificity. However, one of the advantages of cationic polymeric nanoparticles for delivery of DNA to cells is their capacity to deliver large DNA payloads.

DNA dendrimers are complex branched molecules built from interconnected natural or synthetic nucleic acid monomer subunits. Each monomer is composed of two DNA strands that share a region of sequence complementarity located in the central portion of each strand. The two strands anneal to form the monomer, resulting in a structure that has a central double-stranded “waist” bordered by four single-stranded “arms”. The single-stranded “arms” at the ends of different monomer types are designed to base-pair with the “arms” of complementary monomer types. This allows directed assembly of the dendrimer as a step-wise series of monomer layers, beginning with a single initiator monomer. It is a useful feature of dendrimers that addition of the last layer of monomers leaves single-stranded “arms” unpaired on the surface of the molecule. The number of surface layer single-stranded “arms” increases with the number of layers. The single-stranded “arms” on the surface are available for attaching a variety of molecules to provide functionality to the dendrimer, including a label or a molecule specific to a particular application. The functional molecules may be conjugated directly to the dendrimer arm, or attached via hybridization to the dendrimer arm. Because of the multiplicity of available single-stranded “arms,” multiple different functionalities may be attached to the dendrimer, for example a targeting moiety and a label. Dendrimer have been recognized as providing enhanced detection specificity and sensitivity in a variety of assays.

Targeted expression of the diphtheria toxin (DT) gene in specific cells results in death of those cells. Diphtheria toxin is one of the toxins of choice for cell ablation because its mechanism of action is well-known, and the gene has been cloned, sequenced and adapted for expression in mammalian cells. The precursor polypeptide secreted by Corynebacterium diphtheria is subsequently enzymatically cleaved into the A chain and the B chain fragments. The B chain binds to the surface of eukaryotic cells and delivers the A chain into the cytoplasm. DT-A inhibits protein synthesis inside the cell and is extremely toxic; a single molecule is sufficient to kill the cell. The coding sequences for the DT-A subunit have been cloned independently of the DT-B subunit under the control of cell-specific, cis-acting transcriptional regulatory elements to drive expression. After the DT-A subunit kills the cell, it may be released from the dead cells; however, DT-A is not capable of entering neighboring cells.

Thus, there is a need for compositions and methods for targeted delivery of expressible genes to cells which are predictable, specific and provide substantial efficiency in the number of cells that internalize the genes. The present invention addresses these needs.

### SUMMARY

In a first aspect, the invention relates to compositions comprising a DNA dendrimer having one or more DNA sequences linked thereto, the DNA sequences comprising DNA sequences encoding a polypeptide or regulatory RNA linked to DNA sequences that regulate expression of the DNA sequences encoding a polypeptide or regulatory RNA to produce an RNA coding for the polypeptide or to produce the regulatory RNA. Such DNA dendrimers are referred to herein as “functionalized” DNA dendrimers.

In a specific embodiment of the DNA dendrimer having one or more DNA sequences linked thereto, the DNA sequences that regulate expression comprise a promoter.

In a further specific embodiment of the foregoing, the promoter is substantially transcriptionally active only in a specific cell type, for example an epithelial cell, a virally infected cell, a diseased or damaged cell, or a tumor cell. A promoter that is substantially transcriptionally active only in a specific tumor cell may be, for example, a promoter associated with pancreatic cancer (e.g., the mesothelin (MSLN) promoter) or a promoter associated with prostate cancer (e.g., the cytokeratin 5 (K5) promoter). A promoter that is substantially transcriptionally active only in virally infected cells may be, for example, a regulatory sequence of the major early promoter p97 of the HPV16 genome. Alternatively, the promoter that is substantially transcriptionally active in a specific cell type may be a promoter that is substantially transcriptionally active only in the squamocolumnar (SC) junction cells of the cervix, such as the ARG2 gene promoter. This approach may be referred to herein as “targeted gene expression.” The pancreatic cancer-associated promoter may be the MSLN promoter. The prostate cancer-associated promoter may be K5.

In any of the foregoing embodiments, the DNA sequences encoding the polypeptide or regulatory RNA may encode a cytotoxin. In specific examples, the cytotoxin is Diphtheria toxin A.

In any of the foregoing embodiments, the DNA sequences encoding the polypeptide may encode an immunomodulatory protein. In specific examples, the immunomodulatory protein is a cytokine or a chemokine. Further non-limiting examples of cytokines and chemokines include IL-2, IL-6 and RANTES.

In any of the foregoing embodiments, the DNA sequences encoding the polypeptide or regulatory RNA may encode a fluorescent protein. In specific examples, the fluorescent protein is green fluorescent protein (GFP) or cyan fluorescent protein (CFP).

In any of the foregoing embodiments, the DNA sequences encoding the polypeptide or regulatory RNA may encode a regulatory RNA. In specific examples, the regulatory RNA is shRNA, miRNA or pre-miRNA, or siRNA. In a further specific example, the regulatory RNA is a shRNA to indolamine 2,3-dioxygenase (IDO).

In any of the foregoing embodiments, the one or more DNA sequences linked to the DNA dendrimer may comprise DNA sequences encoding a plurality of polypeptides or a plurality of regulatory RNAs. In specific embodiments, the DNA sequences encode plurality of polypeptides, a plurality of regulatory RNAs, or a combination of polypeptides and regulatory RNAs.

In any of the foregoing embodiments, the DNA dendrimer may further comprise a cellular internalization moiety linked thereto. In specific embodiments, the cellular internalization moiety is an antibody that binds to a cell surface protein, a peptide that binds to a cell surface protein, or a ligand that binds to a cell surface receptor. In a particular embodiment, the cellular internalization moiety is an antibody or peptide that binds to the transferrin receptor.

In a further aspect, the invention provides pharmaceutical compositions comprising the DNA dendrimer having one or more DNA sequences linked thereto, the DNA sequences comprising DNA sequences encoding a polypeptide or regulatory RNA linked to DNA sequences which regulate expression of the DNA sequences encoding a polypeptide or regulatory RNA, and a pharmaceutically acceptable carrier. The DNA dendrimer of the pharmaceutical composition may be a functionalized DNA dendrimer according to any of the embodiments discussed above. In specific embodiments, the DNA sequences encoding a polypeptide or regulatory RNA encode a polypeptide or regulatory RNA that is toxic to a cell or tissue. The toxic polypeptide may be, for example, a cytotoxin such as Diphtheria toxin A.

In specific embodiments of the pharmaceutical compositions, the pharmaceutically acceptable carrier for the functionalized DNA dendrimer is a material that targets the composition for local delivery to cells by facilitating direct exposure of the cells to the functionalized DNA dendrimer. For example, a topical delivery vehicle may be used as a carrier for the functionalized DNA dendrimer and applied directly to the desired location to facilitate uptake of the DNA dendrimer by the cells. In a specific embodiment, the topical delivery vehicle may be a composite material, including a mucoadhesive composite material (e.g., a dextran aldehyde polymer mucoadhesive). In a further specific embodiment, the pharmaceutical composition may be topically applied to cervical squamocolumnar junction cells for treatment of HPV16-infected cells.

In a further aspect, the functionalized DNA dendrimers according to any of the foregoing embodiments may be used in methods for treating diseases and conditions of cells such as virally infected cells, cancerous cells, or precancerous cells. In specific embodiments of the methods, the functionalized DNA dendrimer is administered to a patient in need thereof in an amount effective to treat the disease or condition. In specific embodiments, the DNA dendrimer is targeted to the cells by a cellular internalization moiety. Alternatively, or in addition, the DNA dendrimer is targeted to the cells by the carrier of the pharmaceutical composition as described above. The DNA sequences encoding a polypeptide or regulatory RNA will generally be selected for toxicity to the desired cell. The linked DNA sequences which regulate expression of the DNA sequences encoding the polypeptide or regulatory RNA will generally be selected to be substantially transcriptionally active only in the desired cell; however, if a regulatory sequence is deemed not sufficiently transcriptionally specific for the desired cell type, selection of the pharmaceutically acceptable carrier may be used to improve specificity of delivery of the functionalized DNA dendrimer to the desired cell. Alternatively, a DNA dendrimer comprising a non-transcriptionally specific sequence linked to the DNA sequences which encode the polypeptide or regulatory RNA can be targeted to the desired cell using a pharmaceutically acceptable carrier appropriate for local delivery of the composition. Accordingly, this aspect also relates to use of the functionalized dendrimers for treating diseases and conditions of cells such as virally infected cells, cancerous cells, or precancerous cells, wherein the DNA sequences regulating gene transcription are transcriptionally active in the cells and the DNA sequences encoding the polypeptide or regulatory RNA are toxic to the cells.

In a further aspect, the functionalized DNA dendrimers according to any of the foregoing embodiments may be used in methods for identification and/or visualization of HPV infected epithelial cells, comprising topically applying to the HPV infected epithelial cells the pharmaceutical composition of Claim 16, wherein the DNA sequences regulating gene transcription are specifically transcriptionally active in the HPV infected epithelial cells and the encoded polypeptide is detectable in the HPV infected HPV cells. Accordingly, this aspect also relates to use of the functionalized dendrimers for identification and/or visualization of HPV infected epithelial cells, either in vivo, ex vivo, or in vitro, wherein the DNA sequences regulating gene transcription are specifically transcriptionally active in the HPV infected epithelial cells and the encoded polypeptide is detectable in the HPV infected cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a color illustration of the structure of the functionalized DNA dendrimers. FIG. 1B is a color photograph showing the results of the in vitro experiments described in Example 1. FIG. 1C is a color photograph showing the results of the in vivo experiments described in Example 1.

FIG. 2 is a series of color photographs showing the results of the in vivo experiments described in Example 2.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

The phrase “functionalized DNA dendrimer” and related phrases as used herein refers to a DNA dendrimer that has one or more functionally active moieties attached to its surface. Functionally active moieties include, but are not limited to, moieties that target the DNA dendrimer to a particular cell type, moieties that are therapeutically active, moieties that provide detectability to the DNA dendrimer, and moieties that act within a cell to alter a biological or biochemical activity within the cell. Regulatory RNAs are examples of functionally active moieties, and are non-coding RNAs that exert regulatory influences on biological processes in the cell. Such RNAs include miRNAs and siRNAs. Functionalized DNA dendrimers may also be referred to herein as “nanoDNA,” “DNA nanoparticles,” “dendrimer nanoparticles,” and the like.

The terms “toxic”, “toxicity” and similar terms, as used herein in connection with delivery of functionalized DNA dendrimers to cells, refers to the ability of a functionally active moiety attached to the dendrimer to kill the cell.

The phrase “substantially transcriptionally active” only in a particular cell type, “transcriptionally specific” for a specific cell type, or “specifically transcriptionally active,” and related phrases, as used herein, refer to a gene construct that is expressed preferentially in a particular (target) cell type relative to other (non-target) cell types. While 100% specificity of expression is desired, the expression in non-target cells at a low level is not excluded. In contrast, “non-transcriptionally specific” refers to a gene construct that is expressed substantially equally in all or most cells types.

The terms “treat,” “treating” or “treated” and the like, as used herein, refer to reducing or eliminating a disease or condition; reducing or eliminating at least one symptom of a disease or condition, or; preventing a disease, condition or symptom.

The phrase “pharmaceutically acceptable carrier” refers to any suitable vehicle which can be used to formulate DNA dendrimers for administration to a patient to achieve a therapeutic result. The pharmaceutically acceptable carrier itself generally does not provide additional therapeutic effects. The carrier may be adapted for use in intravenous formulations, topical formulations, oral formulations and the like.

The term “promoter” is used herein in its broadest sense, to refer to any regulatory sequence that enhances, induces or regulates transcription of a downstream DNA coding sequence to produce an RNA complementary to the coding sequence (gene expression).

It has now been found that an expressible genetic construct attached to a DNA dendrimer is a useful delivery vehicle for gene therapy which carries large amounts of the gene construct into the cell. It has been found that the DNA dendrimer and the attached gene construct are effectively internalized by the cell upon contact with the cell surface, and that the polypeptide or regulatory RNA of the gene construct (i.e., the gene product) is effectively expressed within the cell. If the gene product is a toxic molecule, its expression kills the cell. DNA dendrimers are further capable of providing enhanced cell specificity for gene construct delivery to cells, by virtue of cell-specific targeting moieties attached to the DNA dendrimer. In addition, use of promoters in the gene construct that are substantially transcriptionally active only in a specific cell type provides an additional level of cell specificity and further improved therapeutic effect.

A first aspect of the invention provides a DNA dendrimer having linked thereto one or more DNA sequences encoding a polypeptide or regulatory RNA (i.e., a coding sequence) operably linked to a DNA sequence that regulates transcription of the coding sequence to produce an RNA coding for the polypeptide or to produce the regulatory RNA (e.g., a promoter). The DNA sequence that regulates transcription of the coding sequence and the operably linked coding sequence together are referred to herein as a “gene construct.” The DNA dendrimer may optionally have further have linked thereto a targeting moiety that directs the DNA dendrimer to a specific cell type and causes binding of the DNA dendrimer to the surface of the specific cell type. This type of DNA dendrimer is adapted for targeted delivery of the gene construct to the specific cell type, where it can be internalized for expression of the coding sequence.

In a further embodiment, the DNA sequences encoding a polypeptide or regulatory RNA are selected such that the encoded polypeptide or regulatory RNA is toxic to a cell upon delivery to the cell via the DNA dendrimer. For example, a DNA sequence encoding DT-A or another cytotoxic polypeptide may be operably linked to the DNA sequence that regulates transcription. In a further example, a DNA sequence encoding a siRNA or miRNA that silences expression of a selected gene in the cell upon delivery to the cell via the DNA dendrimer may be operably linked to the DNA sequence that regulates transcription.

In specific embodiments, the DNA sequence that regulates transcription of the coding sequence is a promoter. Any promoter suitable to obtain expression in the cell upon internalization of the dendrimer is useful in the invention. The promoter may be substantially transcriptionally active only in a specific cell type, or it may be non-transcriptionally specific. The type of promoter will be selected based on the cell type for expression of the gene construct and the desired degree of transcriptional specificity. In general, highly transcriptionally active promoters are preferred to maximize expression of the coding sequence. Examples of relatively non-transcriptionally specific promoters include the HPV16 Long Control Region (LCR) and RG4X (which activate transcription in both HPV16 infected cells and non-infected cells) and CAG (a ubiquitously expressed strong promoter sequence). Examples of promoters substantially transcriptionally active only in specific cell types include the AGR2 promoter (anterior gradient protein 2, which is expressed in the cells of the SC junction), the K5 promoter (expressed in androgen independent prostate cancer cells), and the mesothelin promoter (which is expressed in pancreatic cancer cells.

The DNA sequence that encodes the polypeptide or regulatory RNA may be any DNA sequence that encodes a polypeptide or regulatory RNA that is functionally active in the cell once the DNA dendrimer is internalized. For example, the coding sequence may encode a polypeptide that is toxic to the cell (such as DT-A), a regulatory peptide that suppresses or enhances expression of a gene in the cell, or an enzyme that is functional within the cell. The coding sequence may also encode a regulatory RNA that silences gene expression in the cell, such as snoRNA, shRNA, miRNA, or siRNA. In specific examples these regulatory RNAs may target the RNA transport protein HuR. Alternatively, the coding sequence may encode a regulatory RNA that enhances gene expression in the cell. In a further example, the regulatory RNA may impact developmental processes within the cell, such as the miRNA let7.

In certain embodiments wherein the DNA dendrimer further includes a targeting moiety linked to the single stranded arms. The targeting moiety may be any protein, polypeptide or ligand that binds to a cell surface marker (such as a ligand or receptor) on the cell selected for delivery of the dendrimer. The targeting moiety improves binding of the dendrimer to the cell surface, and thereby facilitates effective systemic administration of the dendrimer. For example, the targeting moiety may be an antibody, a hormone, a receptor ligand or a toxin. Specific examples of useful targeting moieties include those that bind to cell surface markers that are expressed exclusively or almost exclusively on the surfaces of pathological cells or on the surfaces of cells of a particular class, type, organ or tissue. These targeting moieties may bind to HER-2/neu (certain breast cancer types), asialofetuin receptor (hepatocytes), viral antigens (virus-infected cells) or scavenger receptors (macrophages).

Alternatively, useful targeting moieties include those that bind to cell surface markers that are overexpressed on the surfaces of pathological cells or on the surfaces of cells of a particular class, type, organ or tissue. These targeting moieties may bind to PDGF receptor, EGFR family receptors, ICAM, CD receptors, MMP-9, interleukin receptors, transferrin receptor, folic acid receptor, and others known in the art.

If the DNA dendrimer includes a targeting moiety, the targeting moiety may be linked to the dendrimer by a covalent or non-covalent linkage. In a non-covalent linkage, the targeting moiety may be linked to an oligonucleotide that is complementary to a sequence on the single-stranded “arms” of dendrimer, so that the targeting moiety is indirectly hybridized to the dendrimer. Exemplary covalent linkages are known in the art, such as the linkages formed by bifunctional crosslinking agents and hetero bifunctional crosslinking agents, or using “click chemistry”.

In additional aspects, the invention provides pharmaceutical compositions comprising any of the foregoing DNA dendrimers. These compositions comprise the DNA dendrimer and at least one pharmaceutically acceptable carrier in a formulation appropriate for the desired route of administration. The DNA dendrimer is present in the pharmaceutical composition in an amount sufficient to deliver enough gene construct to the cell to treat the identified disease or condition when the dendrimer is internalized by the target cell. The amount of dendrimer in the composition may range from 0.1 to 25 weight % based on the total weight of the pharmaceutical composition. Examples of pharmaceutically acceptable carriers include water, stabilizers, thickeners, lubricants, waxes, chelating agents, surfactants, diluents, anti-oxidants, binders, preservatives, coloring agents (such as pigments or dyes), emulsifiers or other conventional constituents of a pharmaceutical formulation. The pharmaceutical composition may be formulated for intravenous administration, topical administration, injection, infusion, or oral administration as is known in the art. The pharmaceutical composition is typically prepared by combining all ingredients under suitable conditions using methods known in the art.

In further aspects, the invention provides methods of using, and use of, the DNA dendrimers and the pharmaceutical compositions comprising the DNA dendrimers for treating diseases and/or conditions in a patient in need thereof. In general, targeting of the gene construct to the desired cells may be achieved in either of two ways. First, if the promoter is non-transcriptionally specific and the DNA dendrimer does not include a targeting moiety, the pharmaceutical composition comprising the DNA dendrimer may be administered locally to the cells identified for treatment. By way of example, the pharmaceutical composition comprising the DNA dendrimer may be topically applied to the tissue where diseased cells or cells having the condition are found, or it may be administered locally by injection into the tissue where diseased cells or cells having the condition are found. This is referred to as “targeted delivery.” Targeted delivery may also be achieved by a targeting moiety linked to the DNA dendrimer when the targeting moiety binds to a cell type having the disease or condition, but does not substantially bind to normal cells.

Second, if the promoter of the gene construct is substantially transcriptionally active only in cells exhibiting the disease or condition, there is the additional option of systemic administration because, even if the DNA dendrimer is internalized by normal cells, the gene product will not be significantly expressed in the healthy cells. This is referred to as “targeted expression.” Improved systemic delivery of the pharmaceutical composition can be achieved by including both targeted delivery and targeted expression features, as discussed above, in the DNA dendrimer.

In a further aspect, the invention provides methods for making the DNA dendrimers discussed above. Methods for assembly of DNA dendrimers from polynucleotide monomers is known in the art and described, for example, in U.S. Pat. No. 5,487,973 and T. Nilsen, et al. (J. Theor. Biol. (1997) 187: 273-284), the disclosures of which are incorporated herein by reference in their entirety. Targeting moieties, if present, may be covalently or non-covalently linked to the dendrimer using various suitable methods known in the art. Such methods include, for example, linkage via disulfide bonds, N-hydroxysuccinimide (NHS) ester dependent condensation reactions, bifunctional cross-linking, use of polycationic compounds to bridge the moiety to the dendrimer via charge-charge interactions, or direct or indirect hybridization to the dendrimer arms as disclosed in WO2010/017544. Similarly, the gene construct may be covalently or non-covalently linked to the arms of the dendrimer using these methods. Use of capture sequences for linking molecules to dendrimers by hybridization are also generally described by R. Stears, et al. (Physiol. Genomics (2000) 3:93-99). Other references disclosing methods for attaching molecules to dendrimers by hydrogen bonding (hybridization) include Int. Arch. Allergy Immunol. (2012) 157:31-40, BioTechniques (2008) 44:815-818, J. Clin. Microbiol (2005) 43(7):3255-3259, and Small (2009) 5(15):1784-90.

### EXAMPLE 1

### DNA Dendrimer Delivery of a Cytokeratin 5 (K5) Promoter-Regulated Diphtheria Toxin (DT) Suicide Gene

2-layer DNA Dendrimer (2n 3DNA) Preparation: DNA dendrimers were manufactured as previously disclosed (see, e.g., U.S. Pat. Nos. 5,175,270, 5,484,904, 5,487,973, 6,110,687 and 6,274,723, each of which is incorporated by reference in its entirety). Briefly, a DNA dendrimer was constructed from DNA monomers, each of which is made from two DNA strands that share a region of sequence complementarily located in the central portion of each strand. The two strands anneal to form the monomer the resulting structure can be described as having a central double-stranded “waist” bordered by four single-stranded “arms”. This waist-plus-arms structure comprises the basic 3 DNA® monomer. The single-stranded arms at the ends of each of the five monomer types are designed to interact with one another in precise and specific ways. Base-pairing between the arms of complementary monomers allows directed assembly of the dendrimer through sequential addition of monomer layers. Assembly of each layer of the dendrimer includes a cross-linking process where the strands of DNA are covalently bonded to each other, thereby forming a completely covalent molecule impervious to denaturing conditions that otherwise would cause deformation of the dendrimer structure. In addition, 38 base oligonucleotides that serve as complementary capture oligos are ligated to the 5′ ends of available dendrimer arms via a simple T4 DNA ligase dependent ligation reaction, as follows:

Attaching a capture sequence to a DNA dendrimer: To attach the transferrin receptor (TfR/CD71) targeting peptide THRPPMWSPVWP (SEQ ID NO:1) , the capture oligonucleotide was ligated to 10-15% of the dendrimer arms. The oligonucleotide complementary to the capture oligonucleotide was conjugated to the TfR peptide (Bio-Synthesis, www.biosyn.com) or to anti-CD71 antibodies and hybridized in a molar ratio to occupy all of the available capture sequences. Approximately 2-5 peptides or anti-CD71 antibodies were attached per dendrimer molecule as summarized below.

Small (15-100 nucleotides) DNA or RNA capture oligonucleotides (or other biochemical analogs) were covalently attached to the ends of the dendrimer arms via a simple nucleic acid ligation reaction utilizing a bridging oligonucleotide that overlaps adjacent portions of the dendrimer arm and the capture oligonucleotide, thereby bridging the capture oligonucleotide to the end of the dendrimer arm. The bridging oligonucleotide overlapped as least 5 bases of each of the adjacent dendrimer arm and capture oligonucleotide sequences to facilitate the ligation activity of a nucleic acid ligase enzyme (preferably T4 DNA ligase enzyme), with at least 7 bases of overlap of each sequence preferred. The bridging oligonucleotide may also serve as a nucleic acid blocker for its complementary sequences when the dendrimer is used for specific targeting of non-dendrimer nucleic acids or other molecules.

The following components were added to a microfuge tube:
1. 2 layer DNA dendrimer (500 ng/μL) in 1× TE buffer 5.4 μL (2680 ng)
2. a(−)LIG-BR7 Bridging oligo (14 mer) (50 ng/μL) 2.7 μL (134 ng)
3. 10× Ligase buffer 10.2 μL
4. 10× Ligase buffer 10.2 μL
5. Cap03 capture oligo (38 mer) (50 ng/μL) 4.0 μL (200 ng)
6. T4 DNA Ligase (1 U/μL) 10.0 μL (10 units)

The first four reactants were added together, heated to 65° C. and cooled to room temperature. The 5th and 6th reactants were then added and incubated for 45 minutes. The ligation reaction was stopped by adding 2.8 μL of 0.5M EDTA solution. Non-ligated oligonucleotide were removed via the use of 100 k cutoff centrifugal filters (Millipore Corp.), and during the purification washes, the buffer was changed into lx sterile PBS, pH7.4. The capture oligonucleotide is linked to a first single-stranded surface arm of the dendrimer.

In order to target the cell surface and initiate internalization, an antibody or a peptide that binds to the TfR was coupled to the DNA dendrimer. Either antibody to transferrin receptor (TfR/CD71) or a TfR targeting peptide THRPPMWSPVWP (SEQ ID NO:1) were covalently attached to an oligonucleotide that was complementary to the capture oligonucleotide which was previously ligated to the dendrimer (see above). Briefly, the capture oligonucleotide complement (cplCap03), 5′-TTCTCGTGTTCCGTTTGT ACTCTAAGGTGGATTTTT-3′ (SEQ ID NO:2), was covalently coupled using commercial chemistry to anti-CD71 antibody (Solulink) or TfR targeting peptide (Biosynthesis) by the 3′ end and purified by HPLC to remove excess reagents. These conjugates were then hybridized to dendrimer capture oligonucleotides during the final assembly of the reagents (refer to 3 DNA preparation section below). K5/CFP and K5/XX 3 DNA preparation: 700 μg of pK5/CFP (a plasmid containing the promoter of the cytokeratin 5 gene (Krt5) operably linked to the coding sequence of cyan fluorescent protein (CFP)) and 350 μg of pK5/XX (a plasmid containing the promoter of the cytokeratin 5 gene only (i.e., no coding sequence)) were restricted with KpnI-HF (New England Biolabs) to linearize the plasmid, which was then dephosphorylated with Shrimp Alkaline Phosphatase (Affymetrix) in a final volume of 7 mL and 3.5 mL respectively for 2 hours at 37° C. This reaction was subsequently stopped by heating to 80° C. for 10 min. An aliquot of each was removed for gel analysis to confirm digestion of the plasmids. Next, oligonucleotides complementary to a second single-stranded surface arm of the dendrimer were covalently linked to the linearized plasmids using commercial chemistry as described above. Specifically, the hybridization oligonucleotide complementary to the second single-stranded dendrimer arm, 10× ligation buffer and T4 DNA ligase were added to the linearized plasmid to a final volume of 8.4 mL for K5/CFP and 4.2 mL for K5/XX. The ligation was started in a beaker water bath at room temperature which was then put at 4° C. to slow cool and continue overnight. The next morning, an aliquot was removed from each for gel analysis. A Lonza 1.2% agarose gel was run to determine that ligation of the hybridization oligonucleotide to each plasmid was successful. A combination of DNA dendrimer and each ligated plasmid resulted in a shift of the ligated plasmid into the well with the DNA dendrimer, demonstrating successful ligation of the plasmids. The ligated plasmids were then purified using Amicon 100K columns to remove excess hybridization oligos. The final yield of each purified ligated plasmid was determined using the nanodrop.

The labeled oligonucleotides were prepared by attachment of Cy3 to oligonucleotides complementary to a third single-stranded surface arm of the dendrimer, using methods known in the art.

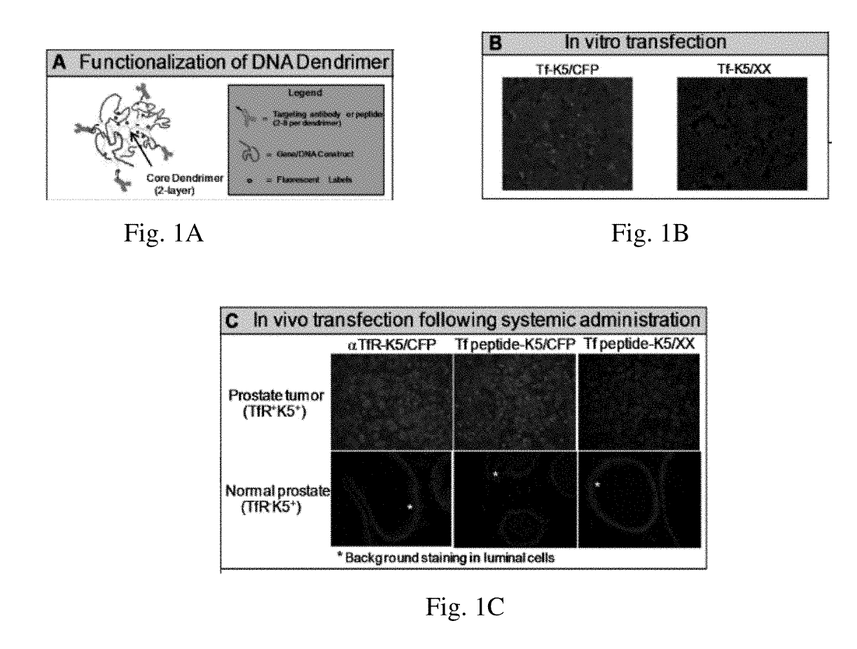

Next, each fully modified 3 DNA dendrimer was prepared by hybridizing the TfR peptide/capture complement (“TfR peptide-cplCap03 conjugate”) or AntiCD71/capture complement (“AntiCD71-cplCap03 conjugate”), the plasmid ligated to the complement of the second single-stranded dendrimer arm (“ligated plasmid”), and the labeled oligonucleotide complementary to the third single-stranded dendrimer arm (“Cy3 label oligo”), to the dendrimer. This was done by combining the following reagents in the order they are listed. For 2n-Cy3 3 DNA-AntiCD71-K5/CFP, 450 μl was prepared to a final concentration of 10 ng/μl 2 n 3 DNA and 489.8 ng/μl plasmid DNA as follows: 4500 ng 2 n (−) 3 DNA, 445 ng c(+) Cy3 label oligo, 192.3 ng AntiCD71 -cplCap03 conjugate, 328.9 μl sterile 1× PBS, and 220.4 μg ligated K5/CFP. For 2n-Cy3 3 DNA-TfR peptide-K5/CFP, 450 μl was prepared to a final concentration of 10 ng/μl 2 n 3 DNA and 489.8 ng/μl plasmid DNA as follows: 4500 ng 2n (−) 3 DNA, 445 ng c(+) Cy3 label oligo, 192.3 ng TfR peptide-cplCap03 conjugate, 347.7 μl sterile 1× PBS, and 220.4 μg ligated K5/CFP. For 2n-Cy3 3 DNA-TfR peptide-K5/XX, 450 μl was prepared to a final concentration of 10 ng/μl 2 n 3 DNA and 489.8 ng/μl plasmid DNA as follows: 4500 ng 2 n (−) 3 DNA, 445 ng c(+) Cy3 label oligo, 192.3 ng TfR peptide-cplCap03 conjugate, 347.7 μl sterile 1× PBS, and 220.4 μg ligated K5/XX. Each 3 DNA dendrimer was incubated at 37° C. for 30 min then stored at 4° C. until use. A diagram of the construct is shown in FIG. 1A.

In Vitro and In Vivo Prostate Tumor Study:

In vitro: To confirm in vitro binding and delivery, 1×10⁵ LAPC4-AS cells (androgen sensitive human prostate cancer cells) were plated in a 24-well plate and cultured at 37° C., 5% CO₂ (balance air) for 2 days. Ten (10) microliters of K5/CFP-TFR (10 ng/ml 3 DNA, 500 ng/ml DNA) or K5/XX-TFR dendrimer (10 ng/ml 3 DNA, 444 ng/ml DNA) was mixed with 100 microliters OptiMEM medium (Gibco). The medium was removed from the 24-well culture plate, and the dendrimer mixture was added and incubated at 37° C. for 4 hrs. One (1) ml of complete medium was added and the culture was continued for 2 days. Cells were immunostained using anti-EGFP antibody (Clonetech 1:1000), and photographed using inverted fluorescent microscope (FIG. 1B).

In vivo: For in vivo testing of the dendrimer constructs, 5×10⁵ PC-3ML/Luc cells (androgen-independent human prostate cancer cells that stably express firefly luciferase) in 100 μl PBS with 20% Matrigel were injected subcutaneously (s.c.) into NOG mice. After 7 weeks, the mice were retro-orbitally injected with 100 μl dendrimer preparation, as prepared above. Twenty-four (24) hours after injection of dendrimer constructs, the mice were sacrificed and tumors, spleen, liver, skin, spleen, kidney, brain, lung and normal prostates were fixed in 10% formalin for 2 hours and embedded in paraffin. Embedded sections were immunostained with anti-GFP antibody (the anti-GFP antibody also recognizes CFP) to determine the level of CFP (Cyan Fluorescent Protein) expression and photographs were taken with a fluorescent microscope. Results are summarized in FIG. 1C. After analyzing the various tissues, it was observed that only the tumor displayed significant expression of CFP, indicating that the dendrimer K5/CFP construct was selectively expressed in tumor cells.

### EXAMPLE 2

### Pancreatic Tumor Targeting

2-layer DNA Dendrimer Preparation: DNA dendrimers were manufactured as previously disclosed (see, e.g., U.S. Pat. Nos. 5,175,270, 5,484,904, 5,487,973, 6,110,687 and 6,274,723, each of which is incorporated by reference in its entirety). Briefly, a DNA dendrimer was constructed from DNA monomers, each of which is made from two DNA strands that share a region of sequence complementarily located in the central portion of each strand. The two strands anneal to form the monomer the resulting structure can be described as having a central double-stranded “waist” bordered by four single-stranded “arms”. This waist-plus-arms structure comprises the basic 3 DNA® monomer. The single-stranded arms at the ends of each of the five monomer types are designed to interact with one another in precise and specific ways. Base-pairing between the arms of complementary monomers allows directed assembly of the dendrimer through sequential addition of monomer layers. Assembly of each layer of the dendrimer includes a cross-linking process where the strands of DNA are covalently bonded to each other, thereby forming a completely covalent molecule impervious to denaturing conditions that otherwise would cause deformation of the dendrimer structure. In addition, 38 base oligonucleotides that serve as complementary capture oligos are ligated to the 5′ ends of available dendrimer arms via a simple T4 DNA ligase dependent ligation reaction, as follows:

Attaching a capture sequence to a DNA dendrimer: To attach the transferrin receptor (TfR/CD71) targeting peptide THRPPMWSPVWP (SEQ ID NO:1), a capture sequence was ligated to 10-15% of the dendrimer arms. The complementary oligonucleotide to this capture sequence was conjugated to the TfR peptide (Bio-Synthesis, www.biosyn.com) and hybridized in a molar ratio to occupy all of the available capture sequences. Approximately 2-5 peptides were attached per dendrimer molecule as summarized below.

Small (15-100 nucleotides) DNA or RNA capture oligonucleotides (or other biochemical analogs) were covalently attached to the ends of the dendrimer arms via a simple nucleic acid ligation reaction utilizing a bridging oligonucleotide that overlaps adjacent portions of the dendrimer arm and the capture oligonucleotide, thereby bridging the capture oligonucleotide to the end of the dendrimer arm. The bridging oligonucleotide overlapped as least 5 bases of each of the adjacent dendrimer arm and capture oligonucleotide sequences to facilitate the ligation activity of a nucleic acid ligase enzyme (preferably T4 DNA ligase enzyme), with at least 7 bases of overlap of each sequence preferred. The bridging oligonucleotide may also serve as a nucleic acid blocker for its complementary sequences when the dendrimer is used for specific targeting of non-dendrimer nucleic acids or other molecules.

The following components were added to a microfuge tube:
1. 2 layer DNA dendrimer (500 ng/μL) in 1× TE buffer 5.4 μL (2680 ng)
2. a(−)LIG-BR7 Bridging oligo (14 mer) (50 ng/μL) 2.7 μL (134 ng)
3. 10× Ligase buffer 10.2 μL
4. Nuclease free water 81.7 μL
5. Cap03 capture oligo (38 mer) (50 ng/μL) 4.0 μL (200 ng)
6. T4 DNA Ligase (1 U/μL) 10.0 μL (10 units)

The first four reactants were added together, heated to 65° C. and cooled to room temperature. The 5th and 6th reactants were then added and incubated for 45 minutes. The ligation reaction was stopped by adding 2.8 μL of 0.5M EDTA solution. Non-ligated oligonucleotide was removed via the use of 100 k cutoff centrifugal filters (Millipore Corp.), and during the purification washes, the buffer was changed into lx sterile PBS, pH7.4. The capture oligonucleotide is linked to a first single-stranded surface arm of the dendrimer.

In order to target the cell surface and initiate internalization, a peptide that binds to the TfR, was coupled to a DNA dendrimer. A targeting peptide THRPPMWSPVWP (SEQ ID NO:1) was covalently attached to an oligonucleotide that is complementary to the capture sequence which was previously ligated to the dendrimer. Briefly, the capture sequence complement (cplCap03), 5′-TTCTCGTGTTCCGTTTGT ACTCTAAGGTGGATTTTT-3′ (SEQ ID NO:2), was covalently coupled using commercial chemistry to TfR targeting peptide (Biosynthesis) by the 3′ end and purified by HPLC to remove excess reagents. These conjugates were then hybridized to dendrimer capture sequences during the final assembly of the reagents (refer to 3 DNA preparation section below). MSLN/DT and MSLN/XX 3 DNA preparation: 1 mg of each of two plasmids (MSLN/DT, containing the promoter of the human mesothelin gene (MSLN) operably linked to the coding sequence of the A chain of Diphtheria toxin, and; MSLN/XX, containing the MSLN promoter with no coding sequence)was restricted with KpnI-HF (New England Biolabs) to linearize the plasmid, and dephosphorylated with Shrimp Alkaline Phosphatase (Affymetrix) in a final volume of 10 mL for 2 hours at 37° C. This reaction was subsequently stopped by heating to 80° C. for 10 min. An aliquot of each was removed for gel analysis to confirm digestion of the plasmid. Next, oligonucleotides complementary to a second single-stranded surface arm of the dendrimer were covalently linked to the linearized plasmids using commercial chemistry as described above. Specifically, the hybridization oligonucleotide complementary to the second single-stranded dendrimer arm, 10× ligation buffer and T4 DNA ligase were added to the linearized plasmid to a final volume of 12 mL. The ligation was started in a beaker water bath at room temperature which was then put at 4° C. to slow cool and continue overnight. The next morning, an aliquot was removed from each for gel analysis. A Lonza 1.2% agarose gel was run to determine that ligation of the hybridization oligonucleotide to the plasmid was successful. A combination of DNA dendrimer and each ligated plasmid resulted in a shift of the ligated plasmid into the well with the DNA dendrimer, demonstrating successful ligation of the plasmids. The ligated plasmids were then purified using Qiagen Endo-free plasmid Maxi Kit and Qiagen tip-500. Two tips were used for each plasmid as the maximum capacity of these tips was 500 μg. After elution from the Qiagen tip-500, there was a final step of ethanol precipitation. Pellets were resuspended with sterile 1× PBS and the final yield of each purified ligated plasmid was determined using the nanodrop.

The labeled oligonucleotides were prepared by attachment of Cy3 to oligonucleotides complementary to a third single-stranded surface arm of the dendrimer, using methods known in the art.

Next, each fully modified 3 DNA reagent was prepared by hybridizing the TfR peptide/capture complement (“TfR peptide-cplCap03 conjugate”), the plasmid ligated to the complement of the second single-stranded dendrimer arm (“ligated plasmid”), and the labeled oligonucleotide complementary to the third single-stranded dendrimer arm (“Cy3 label oligo”), to the dendrimer. This was done by combining the following reagent in the order they are listed. For 2n(−) Cy3 3 DNA-TfR peptide-MSLN/DT, 600 μl was prepared to a final concentration of 10 ng/μl 2n 3 DNA and 317 ng/μl plasmid DNA as follows: 6000 ng 2n (−) 3 DNA, 600 ng c(+) Cy3 label oligo, 256.4 ng TfR peptide-cplCap03 conjugate, 385.2 μl sterile 1× PBS, and 190.2 μg ligated MSLN/DT. For 2n(−) Cy3 3 DNA-TfR peptide-MSLN/XX, 600 μl was prepared to a final concentration of 10 ng/μl 2n 3 DNA and 229 ng/μl plasmid DNA as follows: 6000 ng 2n (−) 3 DNA, 600 ng c(+) Cy3 label oligo, 256.4 ng TfR peptide-cplCap03 conjugate, 425.2 μl sterile 1× PBS, and 137.4 μg ligated MSLN/XX. Each 3 DNA dendrimer mixture was then incubated at 37° C. for 30 min then stored at 4° C. until use.

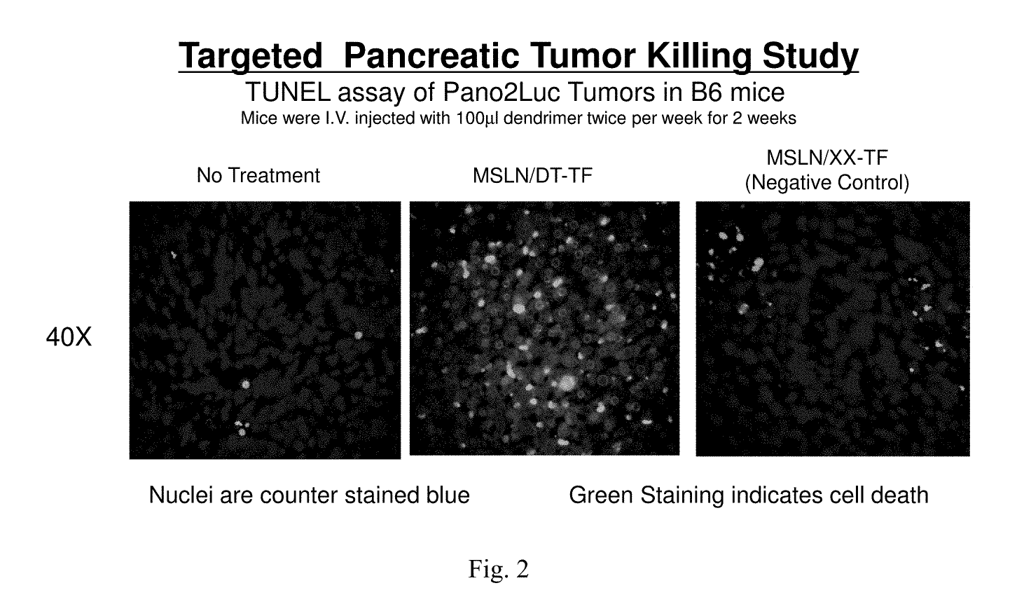

In Vivo mouse study: To induce tumor formation in mice 2×10⁵ PAN02-Luc cells (mouse pancreatic cancer cells that stably expresses firefly luciferase) in 20 ml Matrigel injected directly into the pancreas of B6 mice. Four weeks later, the mice were retro-orbitally injected with 100 μl of 1× PBS (negative control), MSLN/DT dendrimer (10 ng/ml 3 DNA, 311 ng/ml DNA) or with MSLN/XX (10 ng/ml 3 DNA, 229 ng/ml DNA). Injections were made two times per week for 2 weeks for a total of 4 doses. The mice were sacrificed 3 days after the last dose, and the tumor, liver, spleen were fixed in 10% formalin for 2 hours, and paraffin-embedded sections were prepared and mounted on slides for further studies. To determine the level of cell death as a result of the treatment, a TUNEL assay was performed using In situ Cell Death Detection Kit (Roche). The results are summarized in FIG. 2 and comparing the conditions, the MSLN/DT dendrimer construct displayed significantly more cell death than either the PBS buffer control or MSLN/XX (no Toxin gene) control.

### CONCLUSIONS

The transfection data show that functionalized DNA dendrimers deliver K5/CFP DNA to LAPC-4AS cells in vitro and to xenografts following systemic administration. K5/DT kills LAPC-4AI cells in vitro. Because the LAPC-4AI cells were derived from LAPC-4AS cells by growing them in androgen-depleted medium (i.e., simulating androgen depletion therapy (ADT)), cell death upon treatment with K5/DT DNA dendrimers is evidence that ADT and nanoDNA-K5/DT will work together. Targeting the TfR enhances binding of TfR peptide-derivatized dendrimer to TfR-expressing LAPC-4 cells, enhances DNA transfection of these cells in vitro, and enhances DNA expression in TfR-expressing xenografts upon systemic delivery. Non-TfR tissue is negative for binding.

### EXAMPLE 3

### Targeted DNA Dendrimer-Based Gene Therapy for Cervical Precancerous Lesions

Anterior gradient protein 2 (AGR2) was recently identified as a biomarker that is expressed at 25-fold higher levels in squamocolumnar (SC) junctional cells of the cervix as compared to neighboring squamous and columnar cells. A previously identified promoter sequence of the AGR2 gene was identified and shown to have appropriate physiologic activity to target gene expression to SC junctional cells.

2-layer DNA Dendrimer Preparation: DNA dendrimers are manufactured as previously disclosed (see, e.g., U.S. Pat. Nos. 5,175,270, 5,484,904, 5,487,973, 6,110,687 and 6,274,723, each of which is incorporated by reference in its entirety). Briefly, a DNA dendrimer is constructed from DNA monomers, each of which is made from two DNA strands that share a region of sequence complementarily located in the central portion of each strand. The two strands anneal to form the monomer; the resulting structure can be described as having a central double-stranded “waist” bordered by four single-stranded “arms”. This waist-plus-arms structure comprises the basic 3 DNA® monomer. The single-stranded arms at the ends of each of the five monomer types are designed to interact with one another in precise and specific ways. Base-pairing between the arms of complementary monomers allows directed assembly of the dendrimer through sequential addition of monomer layers. Assembly of each layer of the dendrimer includes a cross-linking process where the strands of DNA are covalently bonded to each other, thereby forming a completely covalent molecule impervious to denaturing conditions that otherwise would cause deformation of the dendrimer structure. In addition, 38 base oligonucleotides that serve as complementary capture oligos are ligated to the 5′ ends of available dendrimer arms via a simple T4 DNA ligase dependent ligation reaction.

Attaching a capture sequence to a DNA dendrimer: To attach the transferrin receptor (TfR/CD71) targeting peptide THRPPMWSPVWP (SEQ ID NO:1), a capture sequence is ligated to 10-15% of the dendrimer arms. The complementary oligonucleotide to this capture sequence is conjugated to the TfR peptide (Bio-Synthesis, www.biosyn.com) and hybridized in a molar ratio to occupy all of the available capture sequences. Approximately 2-5 peptides are attached per dendrimer molecule as summarized below.

Small (15-100 nucleotides) DNA or RNA capture oligonucleotides (or other biochemical analogs) are covalently attached to the ends of the dendrimer arms via a simple nucleic acid ligation reaction utilizing a bridging oligonucleotide that overlaps adjacent portions of the dendrimer arm and the capture oligonucleotide, thereby bridging the capture oligonucleotide to the end of the dendrimer arm. The bridging oligonucleotide overlaps at least 5 bases of each of the adjacent dendrimer arm and capture oligonucleotide sequences to facilitate the ligation activity of a nucleic acid ligase enzyme (preferably T4 DNA ligase enzyme), with at least 7 bases of overlap of each sequence preferred. The bridging oligo may also serve as a nucleic acid blocker for its complementary sequences when the dendrimer is used for specific targeting of nondendrimer nucleic acids or other molecules.

The following components are added to a microfuge tube:
1. 2 layer DNA dendrimer (500 ng/μL) in 1× TE buffer 5.4 μL (2680 ng)
2. a(−)LIG-BR7 Bridging oligo (14 mer) (50 ng/μL) 2.7 μL (134 ng)
3. 10× Ligase buffer 10.2 μL
4. Nuclease free water 81.7 μL
5. Cap03 capture oligo (38 mer) (50 ng/μL) 4.0 μL (200 ng)
6. T4 DNA Ligase (1 U/μL) 10.0 μL (10 units)

The first four reactants are added together, heated to 65° C. and cooled to room temperature. The 5th and 6th reactants are then added and incubated for 45 minutes. The ligation reaction is stopped by adding 2.8 μL of 0.5M EDTA solution. Non-ligated oligonucleotide is removed via the use of 100 k cutoff centrifugal filters (Millipore Corp.), and during the purification washes, the buffer is changed into lx sterile PBS, pH7.4. The capture oligonucleotide is linked to a first single-stranded surface arm of the dendrimer.

In order to target the cell surface and initiate internalization, an antibody or a peptide that binds to the TfR, is coupled to a DNA dendrimer. Antibody to transferrin receptor (TfR/CD71) or a targeting peptide THRPPMWSPVWP (SEQ ID NO:1) are covalently attached to an oligonucleotide that is complementary to the capture oligonucleotide which was ligated to the dendrimer. Briefly, the capture oligonucleotide complement (cp1CAP03), 5′-TTCTCGTGTTCCGTTTGT ACTCTAAGGTGGATTTTT-3′ (SEQ ID NO:2), is covalently coupled using commercial chemistry to anti-CD71 antibody (Solulink) or TfR targeting peptide (Biosynthesis) by the 3′ end and purified by HPLC to remove excess reagents. These conjugates are then hybridized to dendrimer capture oligonucleotides during the final assembly of the reagents (refer to 3 DNA preparation section below).

AGR2/DT and AGR2/XX 3 DNA preparation: 1 mg of each of the two plasmids (AGR2/DT, containing the promoter of the human anterior gradient 2 (AGR2) gene operably linked to the coding sequence of the Diphtheria toxin A chain, and; AGR2/XX, containing the AGR2 promoter with no coding sequence) is restricted with a restriction enzyme to linearize it and dephosphorylated with Shrimp Alkaline Phosphatase (Affymetrix) in a final volume of 10 mL for 2 hours at 37° C. This reaction is subsequently stopped by heating to 80° C. for 10 min. An aliquot of each is removed for gel analysis to confirm digestion of the plasmid. Next, oligonucleotides complementary to a second single-stranded surface arm of the dendrimer are covalently linked to the linearized plasmid using commercial chemistry as described above. Specifically, the hybridization oligonucleotide complementary to the second single-stranded dendrimer arm, 10× ligation buffer and T4 DNA ligase are added to the linearized plasmid to a final volume of 12 mL. The ligation is started in a beaker water bath at room temperature which is then put at 4° C. to slow cool and continue overnight. The next morning, an aliquot is removed from each for gel analysis. A Lonza 1.2% agarose gel is run to determine that ligation of the hybridization oligonucleotide to the is successful. A combination of DNA dendrimer and each ligated plasmid results in a shift of the ligated plasmid into the well with the DNA Dendrimer, demonstrating successful ligation of the plasmids. The ligated plasmids are then purified using Qiagen Endo-free plasmid Maxi Kit and Qiagen tip-500. Two tips are used for each plasmid as the maximum capacity of these tips is 500 μg. After elution from the Qiagen tip-500, there is a final step of ethanol precipitation. Pellets are resuspended with sterile 1× PBS and the final yield of each purified ligated plasmid is determined using the nanodrop.

The labeled oligonucleotides are prepared by attachment of Cy3 to oligonucleotides complementary to a third single-stranded surface arm of the dendrimer, using methods known in the art.

Next, each fully modified 3 DNA reagent is prepared by hybridizing the TfR peptide/capture complement (“TfR peptide-cplCap03 conjugate”), the plasmid ligated to the complement of the second single-stranded dendrimer arm (“ligated plasmid”), and the labeled oligonucleotide complementary to the third single-stranded dendrimer arm (“Cy3 label oligo”), to the dendrimer. This was done by combining the following reagent in the order they are listed. For 2n(−) Cy3 3 DNA-TfR peptide-AGR2/DT, 600 μl is prepared to a final concentration of 10 ng/μl 2 n 3 DNA and 317 ng/μl plasmid DNA as follows : 6000 ng 2 n (−) 3 DNA, 600 ng c(+) Cy3 label oligo, 256.4 ng TfR peptide-cplCap03 conjugate, 385.2 μl sterile 1× PBS, and 190.2 μg ligated AGR2/DT. For 2n(−) Cy3 3 DNA-TfR peptide-AGR2/XX, 600 pi is prepared to a final concentration of 10 ng/μl 2 n 3 DNA and 229 ng/μl plasmid DNA as follows: 6000 ng 2 n (−) 3 DNA, 600 ng c(+) Cy3 label oligo, 256.4 ng TfR peptide-cplCao03 conjugate, 425.2 μl sterile 1× PBS, and 137.4 μg ligated AGR2/XX. Each 3 DNA dendrimer mixture is then incubated at 37° C. for 30 min then stored at 4° C. until use.

Ex vivo study: AGR2/DT and AGR2/XX 3 DNA gene expression and cell death is tested following ex vivo treatment of human precancerous cervical tissue with a mucoadhesive material combined with the DNA dendrimers. The mucoadhesive material may be a PEG:dextran composite and/or a composite with thermoreversible properties, such as Lutrol F127. Each sample is placed in a well of a 24-well culture dish, overlaid with the adhesive material containing the DNA dendrimers encoding AGR2/DT and AGR2/XX, and incubated for 24 hr. Optical imaging to detect luciferase bioluminescence and TUNEL assay to detect apoptotic cells is performed.

We expect to observe bioluminescence in samples incubated with adhesive material loaded with AGR2/DT DNA dendrimers, but not in control samples treated with AGR2/XX DNA dendrimers. Similarly, we expect to observe many apoptotic cells in samples incubated with AGR2/DT dendrimers, and few apoptotic cells in tissue treated with control dendrimers.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A composition comprising a DNA dendrimer, wherein one or more linear DNA sequences encoding a cytotoxin operably linked to a promoter having specific transcriptional activity in pancreatic cancer cells or prostate cancer cells are linked to the DNA dendrimer.

2. The composition of claim 1, wherein the promoter is MSLN or K5.

3. The composition of claim 1, wherein the cytotoxin is Diphtheria toxin A chain.

4. The composition of claim 1, comprising a plurality of linked DNA sequences, wherein the linked DNA sequences encode a plurality of cytotoxins.

5. The composition of claim 1, further comprising a cellular internalization moiety linked to the DNA dendrimer.

6. The composition of claim 5, wherein the internalization moiety is an antibody that binds to a cell surface protein, a peptide that binds to a cell surface protein or a ligand that binds to a cell surface receptor.

7. The composition of claim 6, wherein the internalization moiety is an antibody or peptide that binds to the transferrin receptor.

8. The composition of claim 1, wherein the DNA dendrimer is constructed from DNA monomers, each of which comprises two DNA strands having a sequence of complementarity located in a central portion of each strand forming a central double-stranded waist bordered by four single-stranded arms, and the DNA dendrimer is assembled by base pairing between single-stranded arms of complementary monomers in sequential layers, and wherein the one or more linear DNA sequences encoding the cytotoxin are hybridized to available single-stranded arms on a surface of the DNA dendrimer via oligonucleotides complementary to the single-stranded arms ligated to the DNA sequences.

9. A pharmaceutical composition comprising a DNA dendrimer according to claim 1 and a pharmaceutically acceptable carrier.

10. The pharmaceutical composition of claim 9, wherein the pharmaceutically acceptable carrier is a dextran aldehyde polymer mucoadhesive which is a topical delivery vehicle for facilitating uptake of the DNA dendrimer by cells.

11. The pharmaceutical composition of claim 10, wherein the mucoadhesive composite is a PEG:dextran mucoadhesive composite.

12. The pharmaceutical composition of claim 9, wherein the pharmaceutically acceptable carrier is a carrier for systemic administration.

13. The pharmaceutical composition of claim 9, wherein the DNA dendrimer is linked to a cellular internalization moiety.

14. A method for treating pancreatic cancer or prostate cancer in a patient comprising administering to the patient an amount of the DNA dendrimer of claim 1 effective to treat the pancreatic cancer or prostate cancer.
